(19) United States
(12) Patent Application Publication
(10) Pub. No.: US 2018/0243496 A1
(43) Pub. Date: Aug. 30, 2018

(54) DEVICE FOR THE REGIONAL ANTICOAGULATION

(71) Applicant: O2 GROUP SA, Lugano (CH)

(72) Inventors: Michele GAZZANO, Albairate (IT); Marcc INTENNIMEO, Pellezzano (IT)

(21) Appl. No.: 15/760,235

(22) PCT Filed: Sep. 15, 2016
(86) PCT No.: PCT/IB2016/001299

§ 371 Date: Mar. 15, 2018

(30) Foreign Application Priority Data
Sep. 16, 2015 (IT) UB2015A003664

(51) Int. Cl. A61M 1/36; B01D 71/02

(52) U.S. Cl. CPC A61M 1/3675; B01D 71/021

(57) ABSTRACT

A device for regional anticoagulation by citrate-Calcium, of the type usable in a circuit which comprises a first duct or blood line (2) provided with an inlet (1) to withdraw blood from the patient and a downstream outlet (7) for return the treated blood to the patient, device characterized in that it is arranged on said blood line (2) and is provided with a membrane made of a material adapted to bind with the Citrate.

The present invention relates to an apparatus and a circuit for the treatment of blood and relates, in particular, to a device for regional anticoagulation via Citrate Trisodium for extracorporeal circulation treatments.

Over the last years the prevailing trend in the medical field and, in particular, in treatments involving the extracorporeal circulation, is to limit the use of heparin as an anticoagulant to eliminate potentially harmful side effects. The use of heparin has effects on the entire body and is therefore defined as “systemic anticoagulation.”

The use of means that allow to maintain fluid the blood within an extracorporeal circuit but that does not affect the whole patient's organism is defined as “regional anticoagulation.” Among these means the “Calcium-Citrate” system is among the most common.

It consists in adding to the blood taken from the patient, and then very closely to the access point (a catheter or an appropriate needle), a solution containing citrate (generally Citrate Trisodium C₆H₅Na₃O₇) which plays an anticoagulant action.

At the opposite end of the circuit, i.e. immediately before the blood in the extracorporeal circulation is returned to the patient, a suitable solution containing Calcium (Calcium gluconate or Calcium chloride) is added to the blood itself, with the purpose to bring the blood to the natural physiological coagulation conditions.

The addition of the Calcium solution may also be performed to the patient at a different point from the extracorporeal circuit and through independent means.

This system is mainly adopted in hemofiltration machines (dialysis machines for patients in the acute phase) and also in patients undergoing regular dialysis (chronic).

These machines generally employ peristaltic pumps and employ considerable quantities of dialysis and replacement liquid to perform blood purification.

In practice, the blood can be diluted before and/or after the passage through a hemofilter (or artificial kidney) where the dialysis liquid in countercurrent re-establishes the correct ratio of dissolved substances as well as to eliminate the excess water and the waste substances.

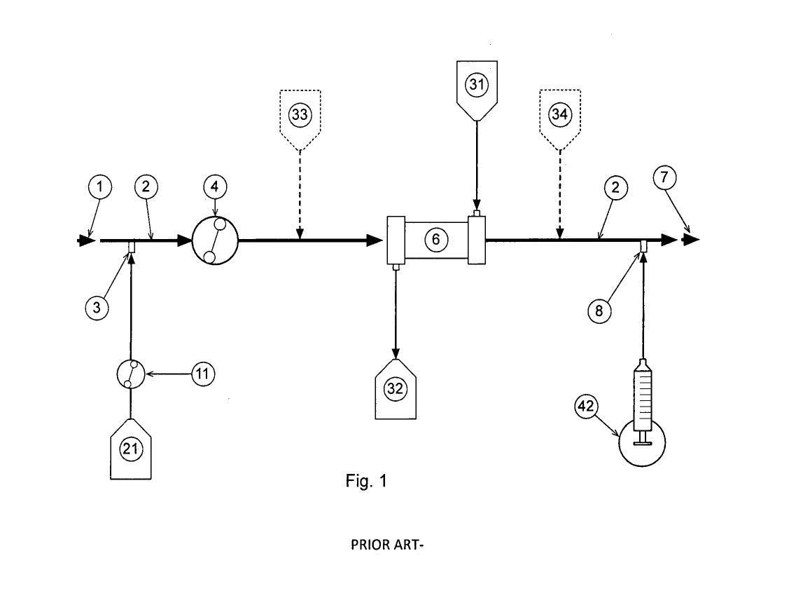

FIG. 1 schematically shows a circuit which operates with this system, in which the citrate is infused by a bag (21) via a peristaltic pump (11) in correspondence of a connection (3) to the blood line (2) arranged upstream the blood pump (4), in the proximity or in the immediate vicinity of the access point (1) that is attached to the patient. In practice, according to the known technique and in general (for example according to procedures known with the abbreviations CRRT, SCUF, HF, CVVH, CVVHV, CVVHVHF, CVVHD, CVVHFD, IVVH and others known to the experts of the sector and that comprise said variants), blood is withdrawn from the end (1) placed upstream of the blood line (2) and is pushed by the pump (4) into the hemofilter (6) where, by means of semi-permeable hollow fibers, it is purified thanks to the contact with the solution withdrawn from a first bag (31) or feeding bag or dialysate bag; the solution coming from the bag (31) together with any predilution liquid of the bag (33), excess water and waste products, are conveyed and collected in a second bag (32) or collection bag or Ultrafiltered bag (UF).

In the diagram of FIG. 1 two optional bags (33) and (34) are represented with a dashed line; these bags are usable for pre-dilution and post-dilution liquids according to the type of treatment to be performed.

The blood thus purified continues along the main line (2) downstream of the hemofilter (6) until the connection (8) where the Calcium solution contained in the syringe pump (42) is mixed to the blood immediately before the point of return to patient (indicated by 7) or, alternatively, inoculated to the patient in another suitable vessel (not shown in this diagram).

Inside of such a treatment it is of little importance the total volume of the solution added from the diluted citrate bag because the total fluid balance is perfectly regulated by the amount of “weight loss” predetermined by the physician and consequently subtracted by the hemofilter and sent to the bag (32).

In the case of extracorporeal treatments that do not provide for a removal of fluid from the patient (as in the case of CO₂ removal treatments) it has so far made use of systemic anticoagulation (heparin) given the difficulty of eliminating from the patient (remarkable) amounts of liquids that are contained in existing solutions for the administration of citrate (also 5 liters per bag) and that are infused with quantity/flows greater than 2.000 ml/h.

The patient who has not a renal failure and is subjected to another extracorporeal treatment such as the extracorporeal removal of CO₂, would be forced to eliminate by means of a natural way (urine) the excess liquid received from the diluted citrate bag; this necessity subjects the kidneys to an excessive workload and could lead to failure in the correct composition of electrolytes and other physiologically substances contained in the patient's blood.

It is important to note that the Citrate which binds to Calcium is still released within the blood circuit and enters the patient's circulatory system where the liver, kidneys and muscles ensure metabolize it to bicarbonate. This involves a considerable workload for the liver in addition to the contraindication to enter large doses of bicarbonate in the human body.

The main object of the present invention is to eliminate the above mentioned drawbacks. This result has been achieved according to the invention by adopting the idea to realize a device able to retain the Ca++ ions from blood with the features described in claim 1.

Other characteristics are object of the dependent claims.

The advantages and features of the present invention will be understood by anyone skilled in the art from the following description and with the help of the attached drawings given as a practical exemplification of the invention, but not to be considered in a limitative sense, wherein:

FIG. 1 illustrates a diagram relating to a circuit according to the prior art, in order to describe a possible example of use of Calcium Citrate-treatment, its particular shape not being limitative of the possible applications of the present invention;

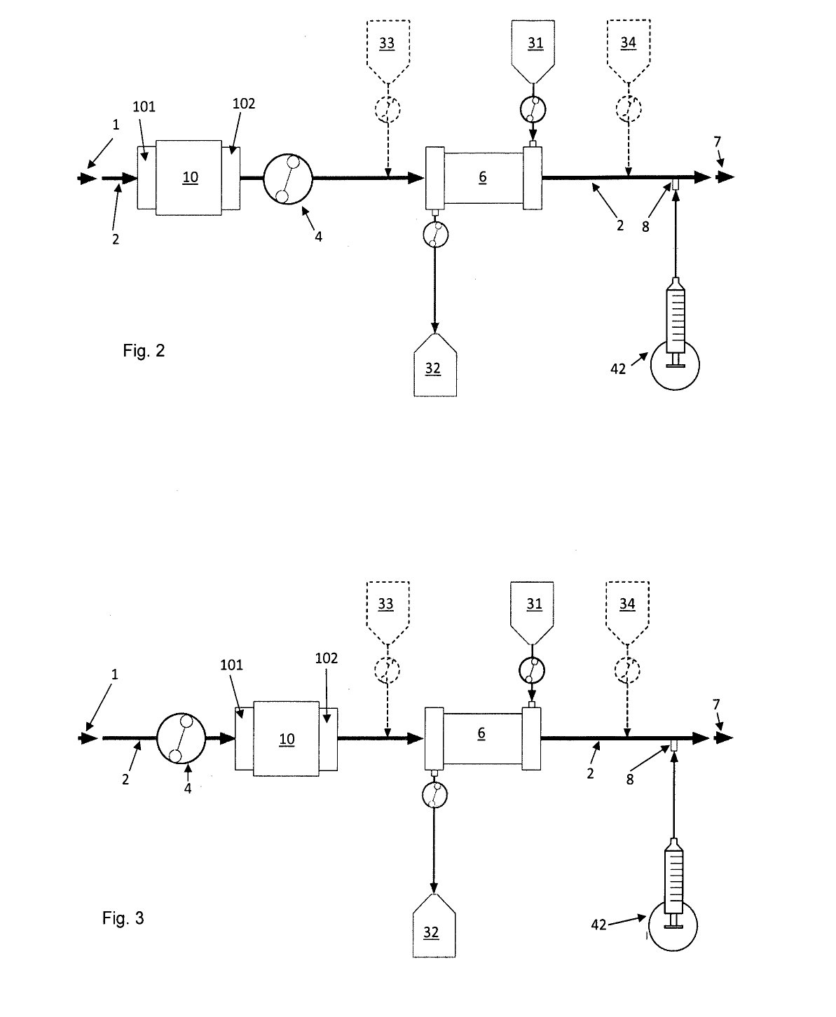

FIGS. 2 and 3 are two possible embodiments of circuits for the treatment of blood achievable according to the present invention; and

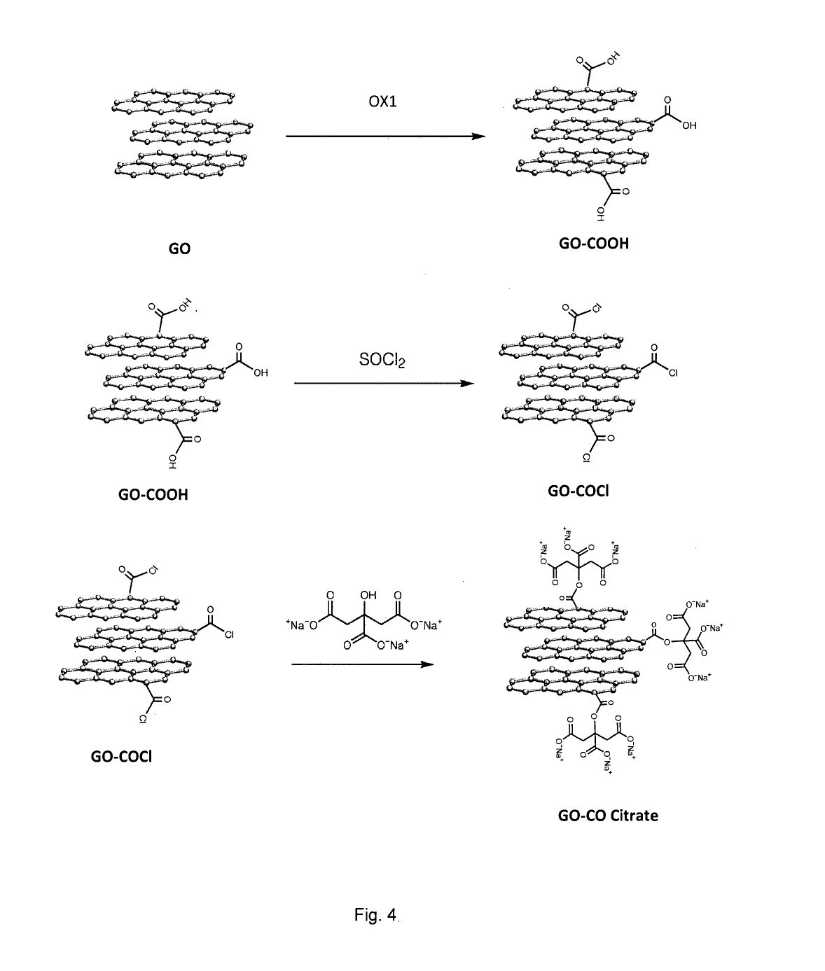

FIG. 4 relates to a diagram to highlight the features of the present invention.

In the present description reference is made to a generic extracorporeal blood treatment with a passage through a hemofilter; this does not exclude that the circuit of the invention can be provided, for example, in place of/or in addition to the hemofilter, with an oxygenator for the removal of CO2, or, in place of the oxygenator or in addition to the same oxygenator, but still in downstream of the device of the present invention (which will be placed in the close proximity of the blood withdrawing point), with any other component as absorbers of cytokines, filters for cholesterol, cartridges for drug delivery, etc. including a circuit for dialysis or hemofiltration.

With reference to the diagram of FIG. 2, a circuit made according to the present invention is usable to obtain the regional anticoagulation by citrate. The numerical references used for many of the components of the circuits of FIGS. 2 and 3 are the same as those of the corresponding components of FIG. 1.

The circuit in question comprises a device (10) which is substantially composed of a container, preferably realized in a biocompatible plastic material, analogous, for example, to that used for the containers of hemofilters and other similar devices. Inside the device (10) is disposed a membrane made from a suitable support substrate which can be realized in graphene, oxidized graphite, alumina or functionalized silica, agar, gel or other suitable support capable of binding with the trisodium citrate by a covalent bond (other supports may include different types of chemical bonds), adapted to retain the citrate and to prevent the same, as well as the complex formed by Calcium Ca++ ions that will bind to such Citrate, has been transported and spread in the blood circuit and then in the patient subjected to the treatment.

Inside this device the blood flows in contact with the surface of the membrane and the calcium ions Ca++ contained in the blood bind to the same Citrate retained by the membrane substrate and therein remained trapped and allowing blood, deprived of such Ca++ ions, to flow freely to the other end of the device.

It therefore gets the result of reducing the concentration of Ca++ ions in the blood in output from the device, thus making the blood does not coagulable and therefore suitable to undergo all subsequent extracorporeal treatments without danger of coagulating.

The main advantage of such a device, compared to the prior art, is to contain and hold inside the citrate and the subsequent Citrate-Calcium compounds preventing their diffusion into the bloodstream. It will be avoided to the patient the need of having to metabolize the Calcium Citrate complex, problematic and difficult at high doses for patients with impaired liver function, simultaneously avoiding the subsequent metabolic transformation into bicarbonate with risk of disorders of various kinds.

In this way the device will contain a predetermined quantity of citrate, associated with an appropriate substrate and it will be able to “chelate”, or to bind, to a pre-determined quantity of Ca++ ions eliminating them from the blood flow.

The device (10) will be realized in different sizes containing different surfaces and quantities of membrane composed by suitable substrate and the citrate bound to it, apt to satisfy the different needs of flow and duration of the various extracorporeal treatments, the same device going in saturation with the subsequent depletion of its anticoagulant properties.

At the end of the extracorporeal treatment it is necessary (as in accordance with the protocols now in use) to return to the patient (for example through the dispenser 42) the calcium that was taken away from him thereby restoring the correct coagulation response. Therefore, according to the present invention, a device for regional anticoagulation by Citrate-Calcium is of the type usable in a circuit which comprises a first duct or blood line (2) provided with an inlet (1) to withdraw the blood from the patient and a downstream output (7) to return the treated blood to the patient; the device (10) is provided with a membrane made of a material suitable to bind with citrate. The device (10) is provided with an input (101) for the blood to be treated and an outlet (102) for the treated blood, i.e. for the blood that has passed through the membrane and was deprived of the citrate.

In particular, the membrane is made of a material suitable to bind with trisodium citrate, in particular covalently.

As previously said, the membrane can be made of a material selected from the group consisting of: graphene, oxidized graphite, alumina or functionalized silica, agar, gel, without, however, excluding other types of supports which are suitable for the purpose. With reference to the example of FIG. 2, the device (10) is arranged upstream of the blood pump (4) which pushes the blood along the line (2) downstream.

In particular, with reference to the non-limiting example represented in FIG. 2, the pump (4) pushes the blood downstream, where on the blood line (2) is arranged a hemofilter (6). As is provided for the example of FIG. 1, the blood is purified in the hemofilter (6), by means of semi-permeable hollow fibers, thanks to the contact with the solution taken from a first bag (31) or feeding bag or dialysate bag; the solution coming from the bag (31) together with any predilution liquid of the bag (33), excess water and waste products, are conveyed and collected in a second bag (32) or the collection bag or Ultrafiltered bag (UF). In the diagram the broken lines represent the two optional bags (33) and (34) and the associated pumps usable for pre-dilution and post-dilution liquids according to the type of treatment to be performed. The blood thus purified continues along the main line (2) downstream of the hemofilter (6) until the connection (8) where the Calcium solution contained in the syringe pump (42) is mixed to the blood immediately before the point of return to patient (indicated by 7) or, alternatively, inoculated to the patient in another suitable vessel (not shown in this diagram).

In FIG. 3 is shown another embodiment of the invention according to which the device (10) is arranged downstream of the pump (4).

The descriptions of the circuits of FIGS. 2 and 3 are to be understood as illustrative and the advantages of the present invention are applicable to any circuit or apparatus in which the regional anticoagulation is performed by Trisodium Citrate, including treatments that do not currently provide for such a system of anticoagulation since they are not able to eliminate the liquid excesses that this practice thus far can not avoid giving.

In FIG. 4 is described in a schematic way a possible principle of the invention. In the first line it is represented a passage which, through oxidation with the Staudenmaier method (OX1), determines in the oxide of graphite CO bonds with oxygen in the form of carboxyl groups (COOH). In the second line is represented a treatment with thionyl chloride (SOCl₂) adapted to transform the peripheral molecules (the carboxylic groups present at the edges) into acyl chlorides (GO—COCl) or in compounds of an acyl group and a chlorine atom. The result is a circle of graphene can react with the Citrate Trisodium, as shown in the third row of FIG. 4.

It is therefore a monolayer of carbon atoms organized structure with a crystalline structure having hexagonal cells: the monoatomic layer is presented as two-dimensional material (2d) that possesses a very large surface (approximately 100-1000 times greater than a typical organic molecule) and that can undergo a large number of reactions on both its sides. The described solution appears to be extremely effective.

The present invention also teaches the implementation of a blood processing procedure which provides for the regional anticoagulation by citrate-calcium, by the use of a circuit that includes a first conduit or blood line (2) provided with an inlet (1) to withdraw blood from the patient and a downstream outlet (7) to return the treated blood to the patient.

According to the process of the invention, the device (10) is provided with a membrane made of a material suitable to bind with citrate. The other characteristics of the membrane are those described previously.

It is understood that the details of execution may vary in any equivalent way as in the shape, dimensions, elements disposition, nature of the used materials, without leaving the scope of the adopted solution and thus remaining within the limits of the protection granted to the present patent.

1. A device for regional anticoagulation by Citrate-Calcium, provided with an input (101) for the blood to be treated and an outlet (102) for the treated blood, characterized in that it is provided with a membrane made of a material able to bind with the Citrate.

2. Device according to claim 1, characterized in that said membrane is made of a material suitable to bind with trisodium citrate.

3. Device according to claim 1, characterized in that said membrane is made of a material to bind with citrate with covalent bond.

4. Device according to claim 1, characterized in that said membrane is made of graphene or oxidized graphite.

5. Device according to claim 1, characterized in that said membrane is made of graphene or oxidized graphite, via oxidation by determining bonds with oxygen in the form of carboxyl (COOH), with subsequent treatment with thionyl chloride (SOCl) adapted to transform Device molecules in acid chlorides (GO—COCl).

6. Device according to clam 1, characterized in that said membrane is made of a material selected from the group consisting of: alumina or functionalized silica, agar, gel having a composition suitable to bind with the Citrate.

7. Extracorporeal blood circuit, comprising a first duct or blood line (2) provided with an inlet (1) to withdraw blood from the patient and a downstream outlet (7) to return the treated blood to the patient, characterized in that it comprises a device (10) disposed on said blood line (2) and provided with a membrane made of a material adapted to bind with the citrate.

8. Circuit according to claim 7, characterized in that it said membrane is made of a material suitable to bind with trisodium citrate.

9. A circuit according to claim 7, characterized in that said membrane is made of a material suitable to bind citrate with a covalent bond.

10. Circuit according to claim 7, characterized in that it said membrane is made of graphene or oxidized graphite.

11. Circuit according to claim 7, characterized in that it said membrane is made of graphene or oxidized graphite, via oxidation by determining bonds with oxygen in the form of carboxyl (COOH), with subsequent treatment with thionyl chloride (SOCl2) adapted to transform Device molecules in acid chlorides (GO—COCl).

12. Circuit according to claim 7, characterized in that said membrane is made of one or more materials selected from the group consisting of, alumina or functionalized silica,
agar, gel having a composition suitable to bind with the Citrate.

13. Circuit according to claim 7, wherein the circuit comprises a blood pump (4) arranged downstream of said inlet (1) and upstream of said outlet (7), characterized in that the device (10) is arranged upstream of said pump (4).

14. Device according to claim 7, wherein the circuit comprises a blood pump (4) arranged downstream of said inlet (1) and upstream of said outlet (7), characterized in that the device (10) is arranged downstream of said pump (4).
